(19) United States
(12) Patent Application Publication
(10) Pub. No.: US 2012/0239003 A1
(43) Pub. Date: Sep. 20, 2012

(54) Multiple Guidewire System

(75) Inventors: Alison J. Julson, Ramsey, MN (US); Mike P. Meyer, Richfield, MN (US); Adam D. Grovender, Brooklyn Park, MN (US)

(73) Assignee: BOSTON SCIENTIFIC SCIMED, INC., Maple Grove, MN (US)

(21) Appl. No.: 13/421,385

(22) Filed: Mar. 15, 2012

Related U.S. Application Data
(60) Provisional application No. 61/452,957, filed on Mar. 15, 2011.

(51) Int. Cl. A61M 25/09

(52) U.S. Cl. 604/528

(57) ABSTRACT

Multiple guidewire systems and methods of using are disclosed. In one illustrative embodiment, a guidewire system may include a first guidewire releasably coupled to a second guidewire such that when the guidewire system is advanced through a vessel, the first guidewire may be coupled to the second guidewire. After the guidewire system has been advanced through a vessel, the first guidewire and the second guidewire may be separated. In some instances, the first guidewire may be chemically, mechanically, electrically, and/or mechanically releasably coupled to the second guidewire.

### CROSS-REFERENCE TO RELATED APPLICATIONS

This application claims the benefit U.S. Provisional Application No. 61/452,957, filed Mar. 15, 2011, the disclosure of which is incorporated herein in its entirety.

### FIELD

The present disclosure relates generally to medical devices, and more particularly, to multiple guidewire systems.

### BACKGROUND

A wide variety of guidewires have been developed for intravascular use. Intravascular guidewires are commonly used in conjunction with intravascular devices such as catheters to facilitate navigation through the vasculature of a patient. Because the vasculature of a patient may be very tortuous, it is desirable to combine a number of performance features in a guidewire. For example, it is sometimes desirable that the guidewire have a relatively high level of pushability and torqueability, particularly near its proximal end. It is also sometimes desirable that a guidewire be relatively flexible, particularly near its distal end. In some medical procedures, multiple guidewires may be used to facilitate the navigation of multiple catheters, or catheters configured to track over multiple guidewires, through the vasculature of the patient. However, when employing multiple guidewires, in some instances, the guidewires may twist and/or cross during advancement through the vasculature making it more difficult to advance a catheter over one or more of the guidewires. Accordingly, there is a need to provide alternative multiple guidewire systems and assemblies to facilitate the navigation of catheters through the vasculature and, in some instances, reduce guidewire crossing.

### SUMMARY

The present disclosure relates generally to medical devices, and more particularly, to multiple guidewire systems. In one illustrative embodiment, a guidewire system may include a first guidewire releasably coupled to a second guidewire such when the guidewire system is advanced through a vessel, the first guidewire is coupled to the second guidewire. When the guidewire system is at a desired location in the vessel, the first guidewire and the second guidewire may be separated. In some instances, the first guidewire is releasably coupled to the second guidewire with a chemical bonding agent (e.g. adhesive), a mechanical coupling (e.g. clamp, interlocking features), a magnetic coupling, and/or an electrical coupling. In some embodiments, the first guidewire and the second guidewire may be longitudinally offset when releasably coupled.

In another illustrative embodiment, a medical system may include a first guidewire and a second guidewire that may be configured to be coupled to the first guidewire when the first and second guidewire are initially advanced through a vessel.

The medical system may also include a first medical device configured to be advanced over the first guidewire to at least partially separate the first guidewire and the second guidewire. In some embodiments, the medical system may also include an adhesive or other chemical bonding agent disposed at least partially between the first guidewire and the second guidewire. The adhesive or other chemical bonding agent may be configured to at least partially dissolve in vivo. In some cases, the medical system may also include a second medical device configured to be advanced over to the second guidewire.

In another illustrative embodiment, a method for guiding a plurality of medical devices to a desired location in a vessel is disclosed. The method may include coupling a first guidewire to a second guidewire, advancing the coupled first guidewire and the second guidewire to the desired location in the vessel, and separating at least a portion of the first guidewire from the second guidewire when the first guidewire and the second guidewire are at the desired location in the vessel. In some embodiments, separating at least a portion of the first guidewire from the second guidewire may include advancing a first medical device over the first guidewire to separate the first guidewire from the second guidewire, pulling the first guidewire and second guidewire apart from outside the vessel, and/or dissolving at least a portion of an adhesive bond between the first guidewire and the second guidewire. The method may also include further advancing at least one of the first guidewire and the second guidewire after the first guidewire is separated from the second guidewire.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION

The disclosure may be more completely understood in consideration of the following detailed description of various illustrative embodiments of the disclosure in connection with the accompanying drawings, in which:

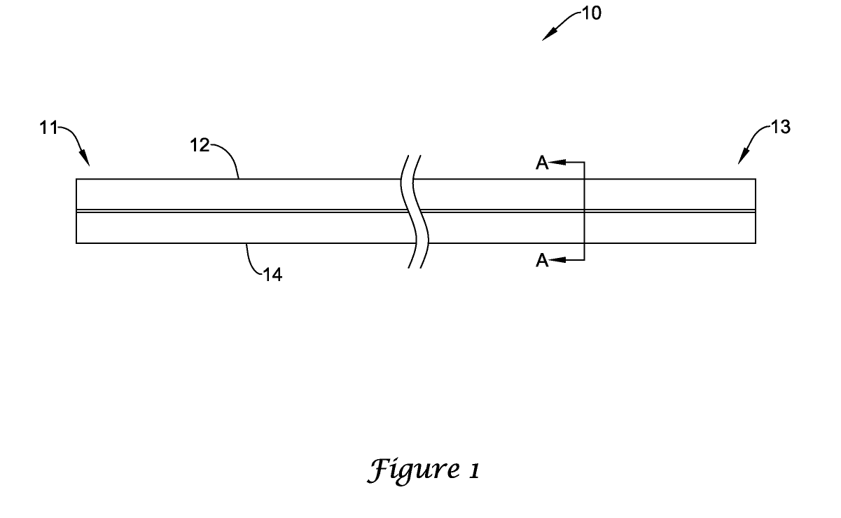

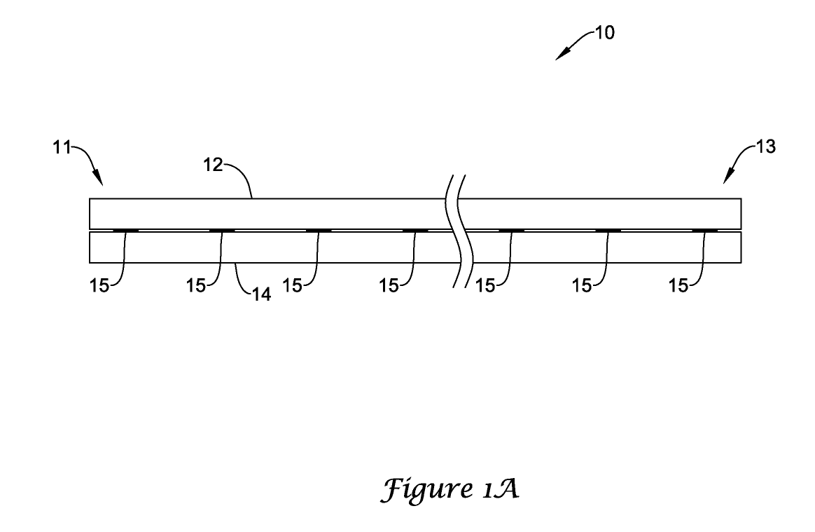

FIGS. 1 and 1A are schematic diagram of an illustrative guidewire system having a first guidewire and a second guidewire;

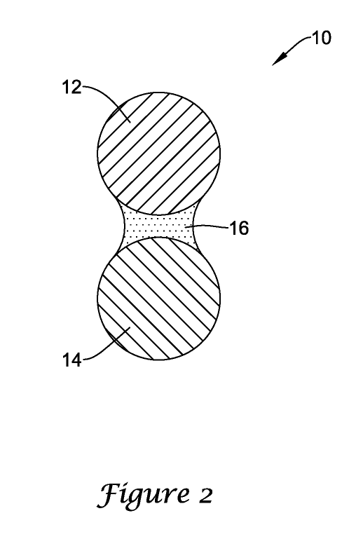

FIG. 2 is a cross-sectional view of the guidewire system of FIG. 1 taken along A-A;

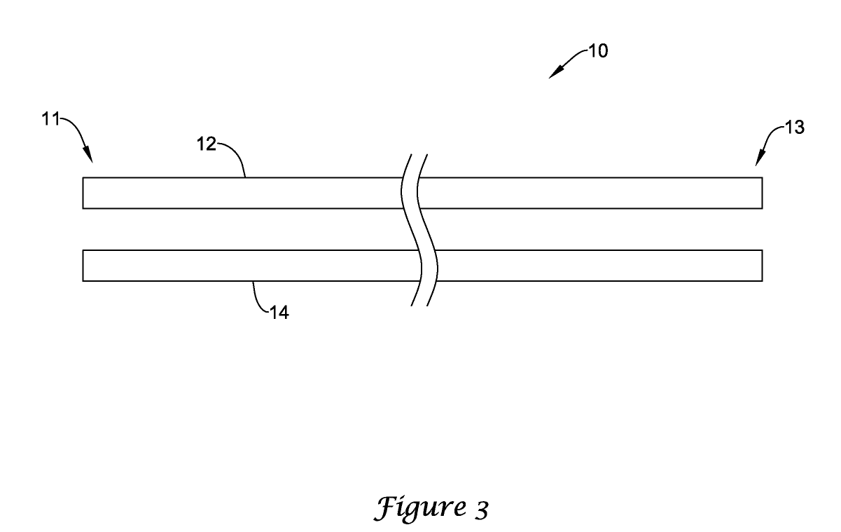

FIG. 3 is a schematic diagram of the illustrative guidewire system of FIG. 1 having the first guidewire separated from the second guidewire;

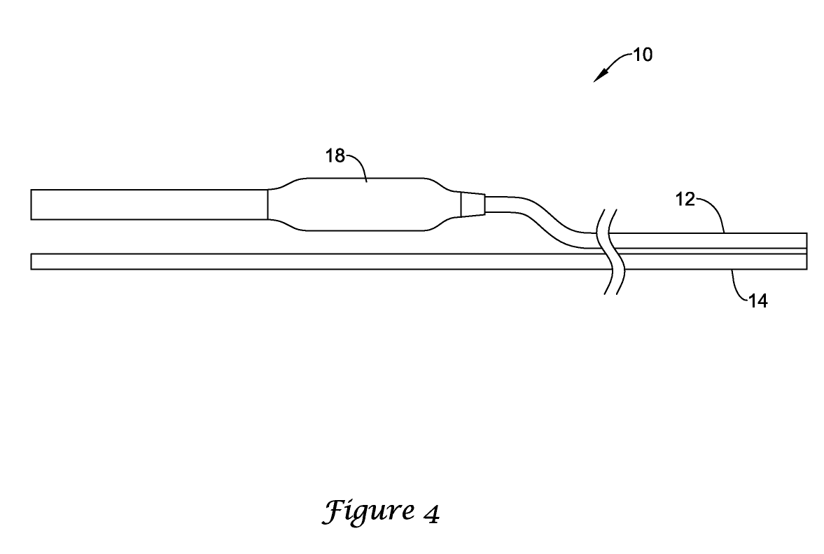

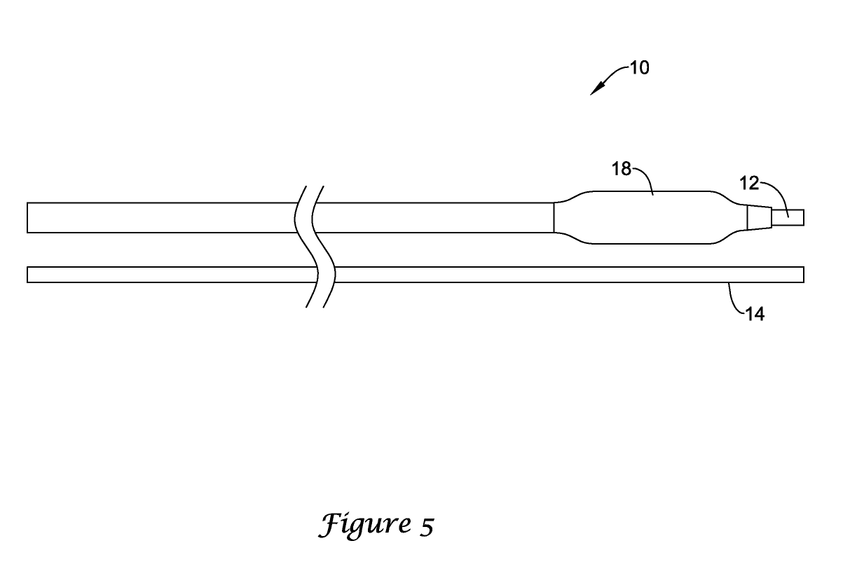

FIGS. 4 and 5 are schematic diagrams of an illustrative method of separating the first guidewire from the second guidewire by tracking a catheter over the first guidewire;

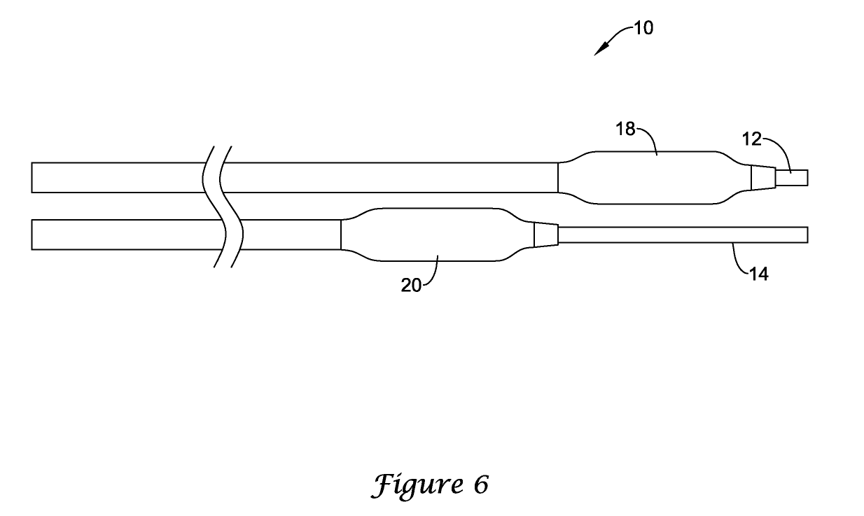

FIG. 6 is a schematic diagram of an illustrative second catheter being tracked along the second guidewire of the guidewire system of FIGS. 4 and 5;

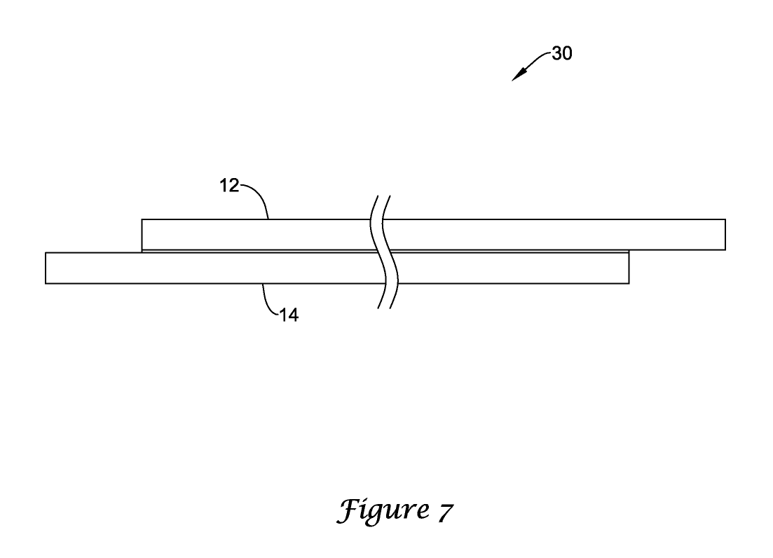

FIG. 7 is a schematic diagram of another illustrative guidewire system having a first guidewire and a second guidewire longitudinally offset;

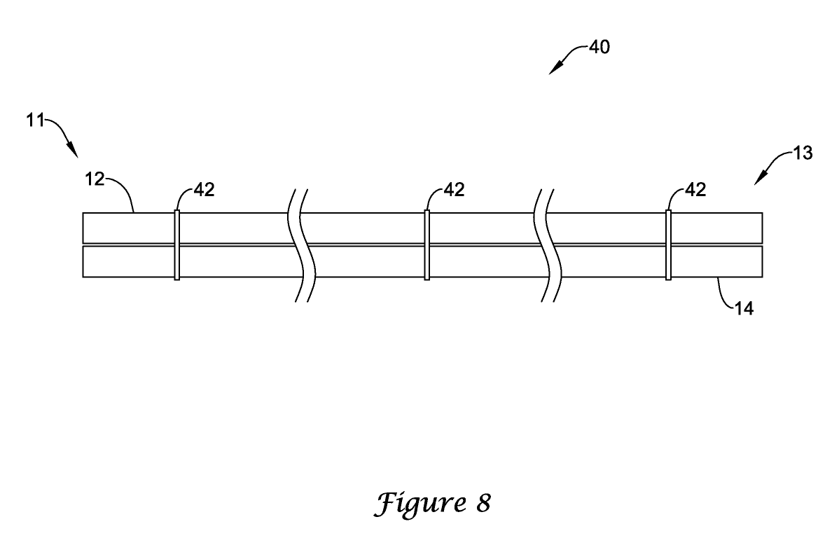

FIGS. 8-10 are schematic diagrams of another illustrative guidewire system employing a mechanical coupling; and

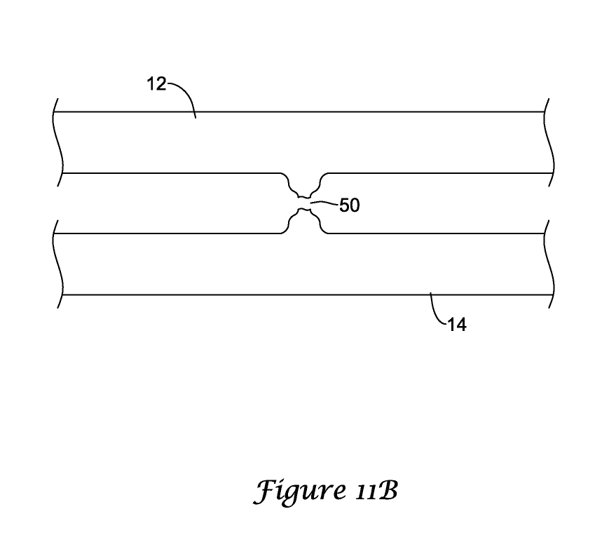

FIGS. 11A-B, 12, 13, and 14 are schematic diagrams of other illustrative mechanical couplings that may be employed to releasably couple the first guidewire to the second guidewire.

### DETAILED DESCRIPTION

The following description should be read with reference to the drawings wherein like reference numerals indicate like elements throughout the several views. The detailed description and drawings show several embodiments which are meant to be illustrative of the claimed invention.

FIG. 1 is schematic diagram of an illustrative guidewire system 10. In the illustrative embodiment, the guidewire system 10 may include a plurality of guidewires, such as guidewires 12 and 14, each including a proximal section 11 and a distal section 13. As shown in FIG. 1, guidewire 12 and guidewire 14 may be releasably coupled, connected, bonded, attached, or otherwise joined together so that guidewires 12 and 14 may be delivered through the vasculature as an assembly or a single unit. With such a configuration, the illustrative guidewire system 10 may help reduce guidewires 12 and 14 from crossing and/or twisting when being advanced through the vasculature of a patient to facilitate that advancement of one or more medical devices through the vasculature. The guidewire system 10 may be configured such that guidewires 12 and 14 may be at least partially separated from one another when positioned at a desired location in the vasculature of the patient.

In the illustrative embodiment, guidewires 12 and 14 may be releasably coupled, connected, bonded, attached, or otherwise joined together with, for example, a chemical coupling, a mechanical coupling, an electrical coupling, a magnet coupling, and/or any other suitable coupling. As shown in FIG. 1, guidewires 12 and 14 may be chemically coupled, or bonded, using an adhesive or other suitable chemical bonding agent. For example, guidewires 12 and 14 may be bonded using adhesive 16 shown in FIG. 2. In some embodiments, the adhesive may be a bioabsorbable and/or a biocompatible adhesive. One example bioabsorbable adhesive is a sugar based adhesive, such as, for example, Mannitol, Xylitol, and/or Sorbitol. However, this is just one example adhesive and it is contemplated that other types of adhesives may be used, as desired.

In some embodiments, guidewires 12 and 14 may be coupled (e.g. bonded) together along an entire length of guidewire 12 and/or 14 (shown in FIG. 1). In other embodiments, guidewires 12 and 14 may be coupled together along only a portion of the length of guidewire 12 and/or 14 (shown in FIG. 1A). That is, guidewires 12 and 14 may be coupled together at spaced apart segments with the chemical, mechanical, electrical, magnetic, or other coupling. As shown in FIG. 1A, guidewires 12 and 14 may be coupled together only at portions 15. In some cases, portions 15 may be configured to be about 20 percent or more, about 35 percent or more, about 50 percent or more, about 60 percent or more, about 75 percent or more, about 90 percent or more, or any other percentage of length, as desired. For example, guidewires 12 and 14 may be coupled together only in the distal section 13 of guidewires 12 and 14 or, in other examples, guidewires 12 and 14 may be coupled (e.g. bonded) together in both the proximal section 11 and the distal section 13. More generally, guidewires 12 and 14 may coupled (e.g. bonded) together in any suitable manner along any portion of length, as desired. When guidewires 12 and 14 are coupled together only in the distal section 13, a guidewire holder or management tool (not shown) may be used to hold the proximal section 11 of guidewires 12 and 14 together.

In some embodiments, guidewires 12 and 14 may be spot welded together at portions 15 with a suitable biodegradable material. For example, guidewires 12 and 14 may be spot welded together with magnesium, which may dissolve when placed in a body vessel for a period of time.

In the illustrative embodiments, guidewires 12 and 14 may be actively and/or passively separated when at a desired location in the vasculature of the patient. For example, guidewires 12 and 14 may be passively separated by allowing the adhesive or other chemical bonding agent to dissolve (or at least partially dissolve) in vivo and/or by expansion of a self-expanding mechanical coupling (e.g. clamp shown in FIG. 8). Also, for example, guidewires 12 and 14 may be actively separated by applying a separation force along at least a portion of the length of guidewires 12 and 14. The separation force may be applied from outside the body or, in other cases, from within the vasculature of the patient by, for example, advancing one or more medical device over one or both of guidewires 12 and 14. In some embodiments, guidewires 12 and 14 may be actively separated by applying or removing an electrical current from one or both of the guidewires 12 and 14. For example, guidewires 12 and/or 14 may be configured to magnetically or electrically attract one another when an electrical potential is applied to or removed from the guidewires 12 and/or 14. Moreover, it is contemplated that a combination of the foregoing separation techniques (active and passive) may be used to separate guidewires 12 and 14. For example, providing a bioabsorbable adhesive that at least partially dissolves may be used in combination with advancing one or more catheters over one or both guidewires 12 and 14 to separate guidewires 12 and 14.

In another embodiment, the guidewires 12 and 14 may be configured to have a chemical attraction that can be reversed and/or released when flushed with a benign chemical. In some cases, the chemical attraction of guidewires 12 and 14 may be induced via a coating applied to the guidewires 12 and 14. It is contemplated that other manners of creating a chemical attraction, which, in some cases, is reversible and/or removable, between the guidewires 12 and 14 may be employed.

In the illustrative embodiment, guidewires 12 and 14 may include any suitable materials, structure, and dimensions according to the desired characteristics and function of the guidewires 12 and 14. For example, the proximal section 11 and the distal section 13 of guidewires 12 and 14 may have a solid cross-section or may have a hollow cross-section. Additionally, proximal and distal sections 11 and 13 may be formed of any suitable material dependent upon the desired properties of the guidewire. Some examples of suitable materials include metals, metal alloys, and polymers. In some embodiments, the proximal section 11 may be formed of relatively stiff material such as straightened 304v stainless steel wire. Alternatively, proximal section 11 may include a metal or metal alloy such as a nickel-titanium alloy, nickel-chromium alloy, nickel-chromium-iron alloy, cobalt alloy, or other suitable material. In general, the material used to construct proximal section 11 may be selected to be relatively stiff for pushability and torqueability.

In some embodiments, the distal section 13 may be formed of a relatively flexible material such as a straightened super elastic or linear elastic alloy (e.g., nickel-titanium) wire, or alternatively, a polymer material, such as a high performance polymer. Alternatively, distal section 13 may be comprised of a metal or metal alloy such as stainless steel, nickel-chromium alloy, nickel-chromium-iron alloy, cobalt alloy, or other suitable material. In general, the material used to construct distal section 13 may be selected to be relatively flexible for trackability.

In some particular embodiments, the distal section 13 may be a linear elastic nickel-titanium alloy, for example, linear elastic nitinol. Within the family of commercially available nitinol alloys, is a category designated “linear elastic” which, although is similar in chemistry to conventional shape memory and superelastic varieties, exhibits distinct and useful mechanical properties. By skilled applications of cold work, directional stress, and heat treatment, the wire is fabricated in such a way that it does not display a “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range. For example, in some embodiments, there is no martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy for the distal portion 16 allows the guidewire to exhibit superior “pushability” around tortuous anatomy.

In some embodiments, the linear elastic nickel-titanium alloy comprises in the range of about 50 to about 60 wt. % nickel, with the remainder being essentially titanium. In some particular embodiments, the composition comprises in the range of about 54 to about 57 wt. % nickel. One example of a suitable nickel-titanium alloy is FHP-NT alloy commercially available from Furukawa Techno Material Co. of Kanagawa, Japan.

In the illustrative embodiment, guidewires 12 and 14 may be configured to have diameters such that one device may be delivered over both guidewires 12 and 14, or in other case, the guidewires 12 and 14 may be configured to have diameters such that a device can be delivered over each guidewires 12 and 14 separately. In some particular embodiments, the proximal section 11 may be formed from a stainless steel wire having a diameter in the range of 0.01 to 0.02 inches, and a length in the range of about 50 to about 110 inches, and the distal section 13 may be formed from a linear elastic nitinol wire having a diameter that ranges from a diameter to match the diameter of the proximal section 11 to as small as about 0.002 inches, and a length in the range of 3 to 15 inches, but this is just one example.

In some embodiments, the proximal section 11 and the distal section 13 may be formed of different materials (i.e., materials having different moduli of elasticity) resulting in a difference in flexibility. For example, the proximal section 11 may be formed of stainless steel wire and the distal section 13 may be formed of nickel-titanium alloy wire, both having the same dimensions, but having a different elastic modulus.

In the illustrative embodiment, guidewires 12 and 14 may include other features typically found in guidewires, such as, for example, one or more tapered and one or more constant diameter regions in the distal tip, a shaping ribbon or wire, and/or radiopaque material, coatings (e.g. lubricious coatings). Some other example coatings and materials and methods used to create such coatings can be found in U.S. Pat. Nos. 6,139,510 and 5,772,609, which are incorporated herein by reference.

In the illustrative embodiment, it is contemplated that guidewires 12 and 14 may include the same or different features and/or materials, as desired. Additionally, while only two guidewire 12 and 14 are shown in FIG. 1, it is contemplated that other numbers of guidewires may be used such as, for example, two or more, three or more, four or more, five or more, six or more, or any other number of guidewires may be used. Example guidewires can be found in U.S. Pat. No. 7,074,197, which is incorporated herein by reference.

FIG. 2 is a cross-sectional view of the guidewire system 10 shown in FIG. 1 taken along A-A. As shown in FIG. 2, the guidewire system 10 may include an adhesive 16 or other chemical bonding agent configured to bond guidewire 12 and guidewire 14 together. As shown, the adhesive 16 or other chemical bonding agent may be applied in the space between guidewires 12 and 14 or, in other words, on corresponding side surfaces of guidewires 12 and 14. However, in other cases, it is contemplated that the adhesive 16 or chemical bonding agent may be applied around one or both guidewires 12 and 14, if desired.

FIG. 3 is a schematic diagram of the illustrative guidewire system 10 of FIG. 1 having guidewire 12 separated from guidewire 14. In the illustrative embodiments, guidewires 12 and 14 may be separated using any of the foregoing active and/or passive separation techniques. For example, guidewires 12 and 14 may be separated by allowing the adhesive 16 or other chemical bonding agent between guidewires 12 and 14 to dissolve (or at least partially dissolve). Additionally or alternatively, guidewires 12 and 14 may be actively separated by, for example, applying a force along at least a portion of the length of the guidewires 12 and 14 by tracking one or more medical device over guidewire 12 or guidewire 14. However, these separation techniques are merely illustrative and it is contemplated that other separation techniques may be used, as desired.

FIGS. 4 and 5 are schematic diagrams of an illustrative method of separating guidewire 12 from guidewire 14 by tracking a catheter 18 over guidewire 12. As shown in FIG. 4, guidewires 12 and 14 may be at least partially manually separated outside of the vasculature of the patient. A proximal end of guidewire 12 may then be inserted into a distal end of catheter 18. The catheter 18 may then be advanced over guidewire 12 applying a separation force to guidewires 12 and 14 pulling them apart.

As shown in FIG. 5, catheter 18 may be further advanced to separate guidewires 12 and 14. In some instances, catheter 18 may be advanced past the distal end of guidewire 12 and/or the distal end to guidewire 14 to completely separate guidewires 12 and 14. Once the guidewires 12 and 14 are separated, one or both of guidewires 12 and 14 may be further advanced in the vasculature of the patient as needed.

In the illustrative embodiment, catheter 18 may be an angioplasty balloon catheter, a stent delivery catheter, and/or any other catheter that is configured to be advanced along a guidewire, as desired. While catheter 18 is shown for merely illustrative purposes, it is not meant to be limiting in any manner. It is contemplated that any suitable medical device may be advanced over guidewire 12 to separate guidewires 12 and 14, as desired.

FIG. 6 is a schematic diagram of an illustrative second catheter 20 being tracked along guidewire 14. As shown, catheter 20 may be advanced along guidewire 14 after the guidewires 12 and 14 have been separated. However, it is contemplated that catheter 20 may be advanced along guidewire 14 before guidewires 12 and 14 are completely separated, if desired.

In the illustrative embodiment, catheter 20 may be an angioplasty balloon catheter, a stent delivery catheter, and/or any other catheter or medical device that is configured to be advanced along a guidewire, as desired.

While FIGS. 4-6 depict two catheters 18 and 20 being advanced over guidewires 12 and 14, it is contemplated that more catheters may be included or that a single catheter having two lumens for tracking along two guidewires may be used. One example catheter having two lumens for tracking along two guidewires may be a bifurcated stent delivery catheter. One example bifurcated stent delivery catheter is disclosed in U.S. Pat. No. 7,655,030, which is incorporated herein by reference.

FIG. 7 is a schematic diagram of another illustrative guidewire system 30. In the illustrative embodiment, guidewire system 30 may be similar to guidewire system 10, except that guidewires 12 and 14 are releasably coupled, connected, bonded, attached, or otherwise joined together in a longitudinally offset configuration. In some embodiments, longitudinally offsetting guidewires 12 and 14 may provide a relatively smaller diameter to a distal end of guidewire system 30 increasing the flexibility of the distal section 13 to improve trackability of the guidewire system 30. In one example, guidewires 12 and 14 may be longitudinally offset or staggered by about 2 to 30 centimeters, about 4 to 25 centimeters, about 8 to 20 centimeters, about 12 to 15 centimeters, or any other longitudinal offset, as desired. In addition to providing increased flexibility to the distal section 13, the longitudinal offset may also allow a user to more easily grasp only one of the guidewires 12 or 14 (e.g. guidewire 14) of the guidewire system 30 in the proximal section 11 while separating the two guidewires 12 and 14.

FIGS. 8-10 are schematic diagrams of another illustrative guidewire system 40. In the illustrative embodiment, guidewires 12 and 14 may include a mechanically coupling to releasably couple guidewires 12 and 14 together. As shown in FIG. 8, the mechanical coupling may include one or more clamps 42, however, any other suitable mechanical coupling may be used.

As shown in FIG. 8, the clamps 42 may be positioned at spaced apart locations along the guidewires 12 and 14 in both the proximal section 11 and the distal section 13. However, it is contemplated that clamps 42 may be provided in only the proximal section 11 or only the distal section 13, if desired. While three clamps 42 are shown, it is contemplated that any number of clamps 42 may be provided including, for example, two or more, three or more, four or more, five or more, ten or more, or any other number of clamps 42, as desired. Additionally, it is contemplated that the size of the clamps may be varied depending on the number of clamps 42 provided and the amount of coupling force desired.

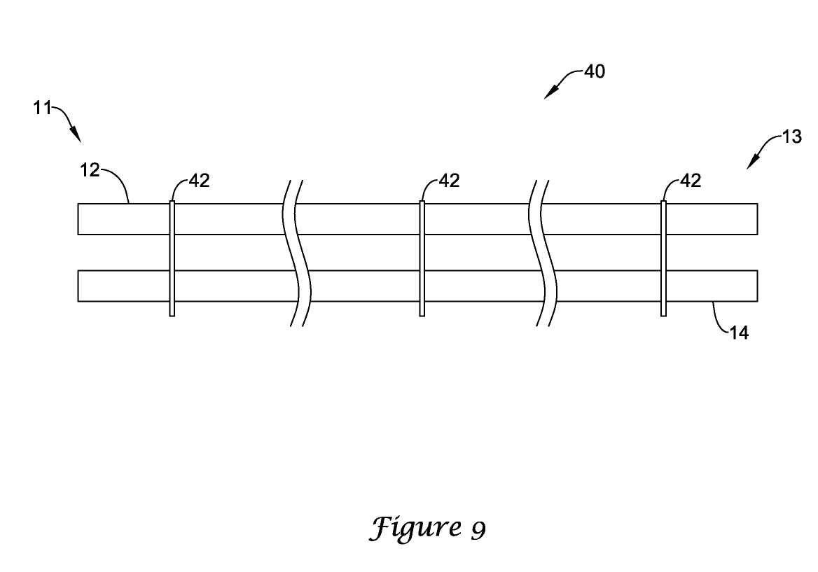

In some embodiments, the clamps 42 may be configured to expand when heated to human body temperature or, in other words, the one or more clamps 42 may include a self-expanding and/or temperature sensitive material One example self-expanding material that may be used is Nitinol, however, other self-expanding and/or temperature sensitive materials may be used, as desired. With such a configuration, the clamps 42 may be configured couple guidewires 12 and 14 together when being advanced through the vasculature of a patient and, once at a desired location in the vasculature, the clamps 42 may radially expand allowing guidewires 12 and 14 to be separated, as shown in FIG. 9.

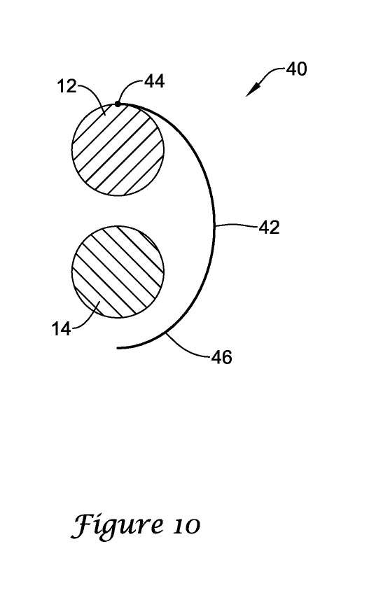

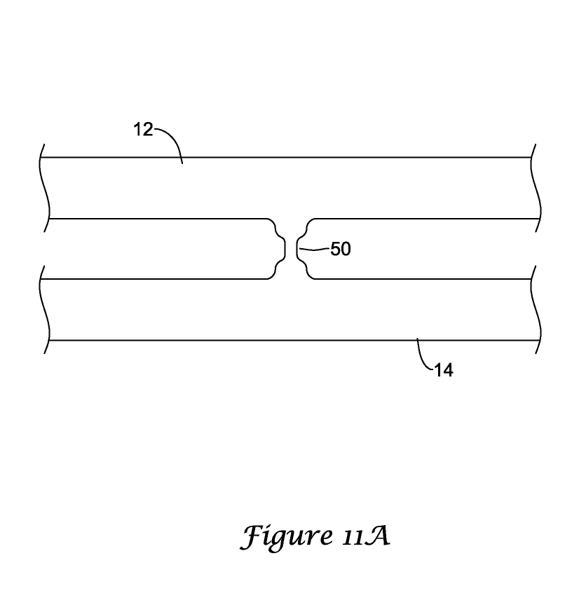

As shown in FIG. 10, the one or more clamp 42 may include a first end 44 attached to guidewire 12 and a second end 46 configured to releasably engage guidewire 14. When clamp 42 is expanded, as shown in FIG. 10, the second end 46 of clamp may disengage guidewire 14 allowing guidewires 12 and 14 to separate. In some embodiments, clamp 42, and in particular first end 44, may be relatively flexible and/or pliable to allow one or more catheters to be advanced over guidewire 12.

In other embodiments, clamps 42 may be configured to dissolve when positioned in a body vessel for a period of time. One example material that may be used for such clamps is magnesium. However, other suitable materials may be used.

FIGS. 11A and 11B are schematic diagrams of an illustrative mechanical coupling between guidewires 12 and 14. As shown in FIG. 11A, a piezoelectric structure 50 may be fabricated on guidewires 12 and 14 to mechanically connect guidewires 12 and 14. The piezoelectric structure 50 may be configured so that upon exposure to a sufficient electrical potential, piezoelectric structure 50 fail and/or break as shown in FIG. 11B separating guidewires 12 and 14. For example, the piezoelectric structure 50 may change shape with an applied current, which may in turn cause the structure to break. One example piezoelectric structure 50 may be gold bonds, however, other piezoelectric structures may be used.

Furthermore, piezoelectric structure 50 shown in FIGS. 11A and 11B is merely illustrative and is not meant to be limiting in any manner. It is contemplated that other suitable piezoelectric structures may be used to connect guidewires 12 and 14, as desired.

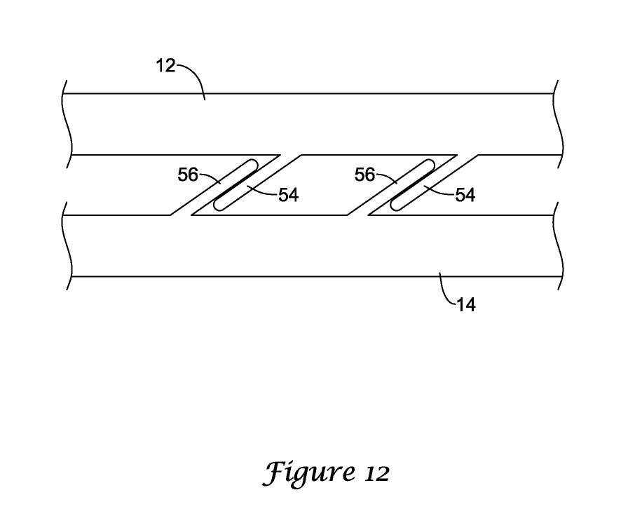

FIG. 12 is a schematic diagram of another illustrative mechanical coupling for guidewires 12 and 14. In the illustrative embodiment, the mechanical coupling may include one or more protrusions 54 extending radially outward from guidewire 12 and one or more protrusions 56 extending radially outward from guidewire 14. In the illustrative example, protrusions 54 may be configured to engage and interlock with protrusions 56 to maintain guidewires 12 and 14 coupled together when being advanced through the vasculature of the patient.

In the illustrative embodiment, relative movement of guidewires 12 and 14 may cause protrusions 54 and 56 to engage and disengage, as desired. For example, applying a force to guidewire 12 in a relative right-to-left direction (as shown) may cause protrusion 54 to engage protrusion 56 whereas applying a force on guidewire 12 in the opposite direction (left-to-right as shown) may cause protrusion 54 to disengage protrusion 56 allowing guidewires 12 and 14 to separate.

While protrusions 54 and 56 are shown, these are just one example. It is contemplated that any suitable protrusions, recesses, and/or combination of protrusions and recesses may be used to create a mechanical coupling or an interlocking structure for releasably coupling guidewires 12 and 14.

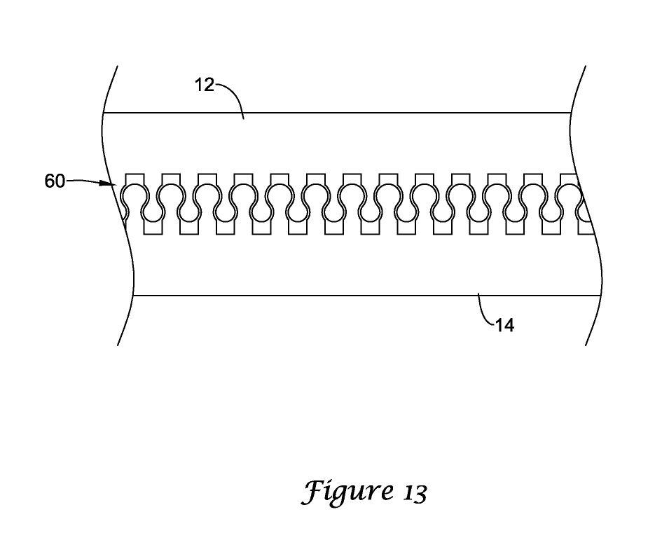

FIG. 13 is a schematic diagram of another illustrative mechanical coupling between guidewires 12 and 14. In the illustrative embodiment, the mechanical coupling 60 may be a zipper-like structure. In the illustrative embodiment, guidewires 12 and 14 may be advanced through the vasculature of the patient in a “zipped” configuration. Once at a desired location in the vasculature, the guidewires 12 and 14 may be “unzipped” to separate guidewires 12 and 14. In some cases, mechanical coupling may be “unzipped” by advancing a device (e.g. catheter) over one or both of guidewires 12 and 14. However, other suitable methods may be used to “unzip” mechanical coupling 60, as desired.

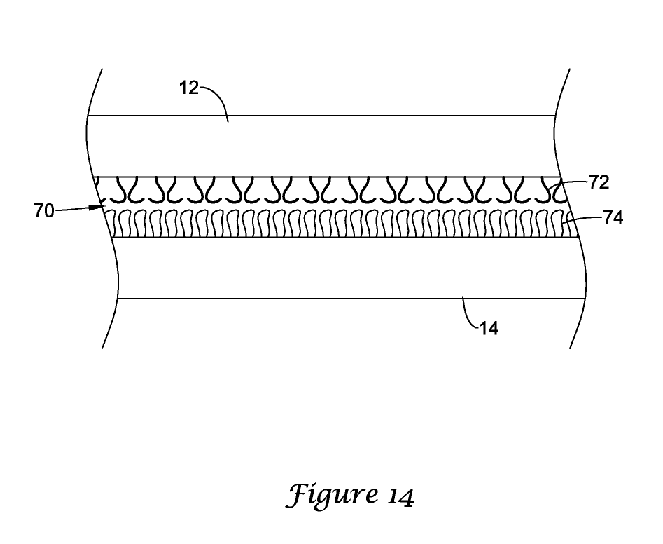

FIG. 14 is a schematic diagram of another illustrative mechanical coupling between guidewires 12 and 14. In the illustrative embodiment, the mechanical coupling may include Velcro 70, such as micro-Velcro, or other type of hook-and-loop fastener. As shown, hooks 72 of the Velcro 70 may be attached to guidewire 12 and loops 74 of the Velcro 70 may be attached to guidewire 14. However, other arrangements may be provided including, for example, hooks 72 being attached to guidewire 14 and loops 74 being attached to guidewire 12, or each guidewire 12 and 14 having alternating portions of hooks 72 and loops 74 that are configured to mate with corresponding loops 74 and hooks 72 on the other guidewire. For illustrative purposes, hooks 72 and loops 74 are shown as being separated.

In the illustrative embodiment, guidewires 12 and 14 may be advanced through the vasculature of the patient in a connected configuration. Once at a desired location in the vasculature, the guidewires 12 and 14 may be separated by either advancing a device (e.g. catheter) over one or both of guidewires 12 and 14 or pulling apart guidewires 12 and 14. However, other suitable methods may be used to separate Velcro 70, as desired.

Having thus described many embodiments of the present disclosure, those of skill in the art will readily appreciate that yet other embodiments may be made and used within the scope of the claims hereto attached. Numerous advantages of the disclosure covered by this document have been set forth in the foregoing description. It will be understood, however, that this disclosure is, in many respect, only illustrative. Changes may be made in details, particularly in matters of shape, size, and arrangement of parts without exceeding the scope of the disclosure. The invention's scope is, of course, defined in the language in which the appended claims are expressed.

What is claimed is:

1. A guidewire system comprising:
a first guidewire including a proximal region and a distal region; and
a second guidewire including a proximal region and a distal region;
wherein the first guidewire is releasably coupled to the second guidewire such when the guidewire system is being advanced through a vessel, the first guidewire is coupled to the second guidewire and, when the guidewire system is at a desired location in the vessel, the first guidewire is at least partially separated from the second guidewire.

2. The guidewire system of claim 1, wherein the first guidewire is releasably coupled to the second guidewire with a chemical coupling.

3. The guidewire system of claim 2, wherein the chemical coupling includes an adhesive.

4. The guidewire system of claim 3, the adhesive is a bioabsorbable adhesive that is configured to at least partially dissolve in vivo.

5. The guidewire system of claim 1, wherein the first guidewire is releasably coupled to the second guidewire with a mechanical coupling.

6. The guidewire system of claim 5, wherein the mechanical coupling includes at least one clamp configured to couple the first guidewire to the second guidewire.

7. The guidewire system of claim 6, wherein the at least one clamp includes a self-expanding material.

8. The guidewire system of claim 6, wherein the at least one clamp includes a first end attached to the first guidewire, wherein the first end is relatively flexible and pliable such that a medical device can be advanced over the at least one clamp.

9. The guidewire system of claim 5, wherein the mechanical coupling includes one or more interlocking features coupled to first guidewire and/or the second guidewire.

10. The guidewire system of claim 1, wherein the first guidewire is releasably coupled to the second guidewire with a magnetic coupling.

11. The guidewire system of claim 1, wherein the first guidewire and the second guidewire are configured to be separated when an electrical potential is applied to the first guidewire or the second guidewire.

12. The guidewire system of claim 1, wherein the first guidewire and the second guidewire are longitudinally offset when releasably coupled together.

13. A medical device system comprising:
two or more guidewires coupled together for advancement through a vessel; and
a first medical device configured to be advanced over a first guidewire of the two or more guidewires, wherein the first medical device at least partially separates the two or more guidewires when being advanced over the first guidewire.

14. The medical device system of claim 13, further comprising an adhesive disposed at least partially between the two or more guidewires, wherein the adhesive is configured to at least partially dissolve in vivo.

15. The medical device system of claim 13, further comprising a second medical device configured to be advanced over to a second guidewire of the two or more guidewires.

16. A method for guiding a plurality of medical devices to a desired location in a vessel, the method comprising:
coupling two or more guidewires together;
advancing the two or more guidewires to the desired location in the vessel; and
separating at least a portion of a first guidewire of the two or more guidewires from a second guidewire of the two or more guidewires when the two or more guidewires are at the desired location in the vessel.

17. The method of claim 16, wherein separating at least a portion of the first guidewire from the second guidewire includes advancing a first medical device over the first guidewire to separate the first guidewire from the second guidewire.

18. The method of claim 16, wherein separating at least a portion of the first guidewire from the second guidewire includes pulling the first guidewire and second guidewire apart from outside the vessel.

19. The method of claim 16, wherein separating at least a portion of the first guidewire from the second guidewire includes dissolving at least a portion of an adhesive bond between the first guidewire and the second guidewire.

20. The method of claim 16, further comprising further advancing at least one of the first guidewire and the second guidewire after the first guidewire is separated from the second guidewire.
